(19) United States
(12) Patent Application Publication
(10) Pub. No.: US 2019/0336653 A1
(43) Pub. Date: Nov. 7, 2019

(54) COATED STENT WITH SURFACE STRUCTURE OR PLACEHOLDER MATERIAL TO REDUCE CRACK FORMATION

(71) Applicant: Biotronik AG, Buelach (CH)

(72) Inventors: Robert Kay, Rostock (DE); Christoph Forkmann, Rostock (DE); Ullrich Bayer, Bad Doberan (DE)

(21) Appl. No.: 16/388,138

(22) Filed: Apr. 18, 2019

(30) Foreign Application Priority Data
May 3, 2018 (DE) 102018110582.7

(51) Int. Cl. A61L 31/10; A61F 2/915; A61L 31/14

(52) U.S. Cl. CPC A61L 31/10; A61F 2/915; A61F 2002/91583; A61F 2250/001; A61L 31/145

(57) ABSTRACT

A stent has a base body coated with a coating and extending in an axial direction, the base body has a plurality of mutually connected struts, each strut having at least one curved section which has a concave side and a convex side, the stent being expandable from an initial state into an expanded state, a curvature of the curved section being reduced in the expanded state compared to the initial state, and a surface structure on the concave side and/or a layer of a placeholder material between the coating and the curved section.

### PRIORITY CLAIM

This application claims priority under 35 U.S.C. § 119 and all applicable statutes and treaties from prior German Application DE 10 2018 110 582.7, filed May 3, 2018.

### FIELD OF THE INVENTION

The invention relates to a stent, and in particular to a biodegradable stent, having a base body that is formed of a magnesium alloy and coated with a coating, such as a polymer or copolymer.

### BACKGROUND

Such stents are used, for example, to keep a constricted vessel of a patient open or to support the surrounding vascular wall and can be implanted via conventional techniques, for example with balloon catheter or via self-expansion. Application include implantation in the peripheral, coronary, cranial and renal areas and in the Eustachian tube.

In addition to releasing a drug, the coating on such a stent, sometimes referred to as a scaffold, fulfills another function: by impeding the exchange of ions between the blood and the magnesium of the base body, the degradation rate of the stent is reduced. Cracks frequently occur in the coating during the dilation of scaffolds, thereby impairing this protective action, which increases the degradation rate at least locally, ultimately thereby reducing scaffolding time. The development of cracks is very likely attributable to mechanical stresses occurring in the coating, in particular on the insides or on the concave sides of the arched or curved sections of the stent. During dilation, the stresses exceeding the plastic deformation capability of the coating. Possible local adhesions of the coating on the scaffold surface further reinforce this problem.

Existing approaches are aimed, for example, at limiting the permissible dilation diameter or developing more resilient polymers for coating the base body.

If the formation of cracks in the coating or in the polymer were merely tolerated, which could, in general, be considered as an option, this is instantaneously associated with undesirably reduced scaffolding time, during which the potential of the implant is not fully utilized.

Limiting the dilation diameter will result in the material boundaries of the magnesium material of the base body, which are already comparatively low, to be utilized even less and increases the risk of applying excessive stress to the scaffold (in particular to the coating).

The provision of more resilient polymers for the coating requires considerable regulatory complexity during the product approval process. In addition, mechanical resilience constitutes only one of many requirements with regard to such a coating polymer and can therefore not be arbitrarily increased.

### SUMMARY OF THE INVENTION

A stent comprising a base body coated with a coating and extending in an axial direction, the base body comprising a plurality of mutually connected struts, each strut comprising at least one curved section which has a concave side and a convex side, the stent being expandable from an initial state into an expanded state, a curvature of the curved section being reduced in the expanded state compared to the initial state, and a surface structure on the concave side and/or a layer of a placeholder material between the coating and the curved section. The likelihood of cracking is reduced compared to conventional approaches described above.

### BRIEF DESCRIPTION OF THE DRAWINGS

Further features and embodiments of the present invention will be described hereinafter based on the figures. In the drawings:

FIG. 1 shows electron microscopic images (left) and FE simulations (right) of the coating on dilated magnesium stents;

FIG. 2 shows electron microscopic images of electropolished magnesium stents having a rough surface. The roughness is caused by intermetallic compounds of the alloying elements with magnesium. These come to the surface during electropolishing and cause undesirably high adhesion to the coating to be applied thereafter (polymer layer);

FIG. 3 shows the tensile stresses generated in the coating by the deformation of the base body of the stent: tension along the concave side or flank (A), tension across the strut width (B);

FIG. 4 shows a schematic representation of an embodiment of a stent according to the invention having an undulated surface or contour on the concave side of the respective curved section or meander curve of the base body so as to create a coating having undulations as a strain reserve;

FIG. 5 shows a schematic representation of a curved section of a base body of an embodiment of a stent according to the invention, wherein the placeholder material is initially arranged on the convex side of the curved section;

FIG. 6 shows a schematic representation of a curved section of a base body of a further embodiment of a stent according to the invention, wherein the placeholder material is initially distributed on all sides of the curved section; and

FIG. 7 shows a perspective view of an embodiment of a stent according to the invention.

### DETAILED DESCRIPTION OF THE PREFERRED EMBODIMENTS

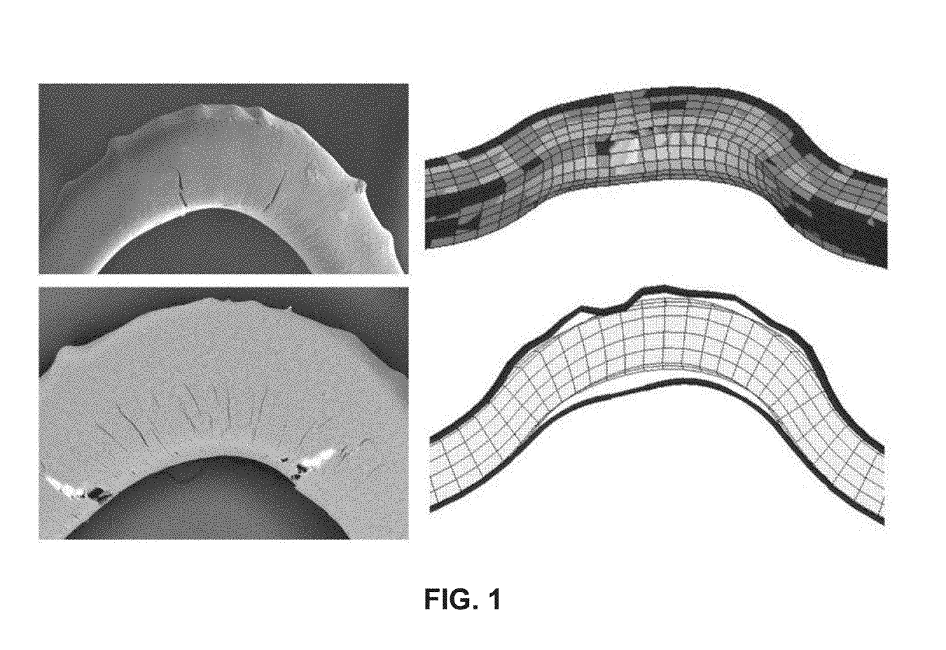

The inventors have determined vulnerable regions and likely causes for cracking of coatings on stents. Electron microscopic images show that the cracks occur primarily in the region of the concave cutting flank of the meander curves (see left of FIG. 1). FE simulations of the dilation behavior (see right of FIG. 1) identify critical areas there where high tensile stress is present in the coating or polymer, which presumably results in the aforementioned cracks.

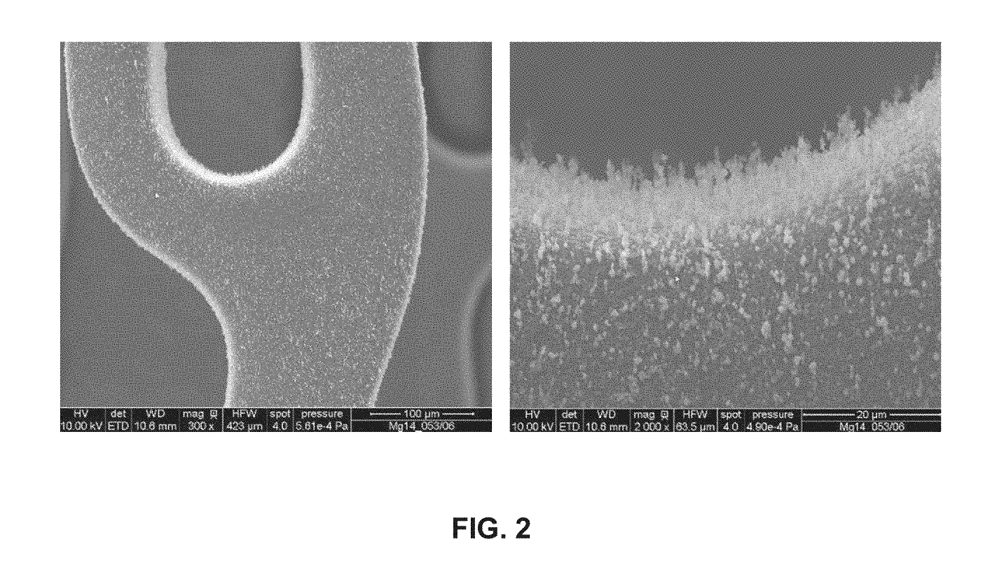

FIG. 2 further shows an extreme example of a rough scaffold surface in which undesirably strong adhesion of the polymer layer is to be expected. Since the coating or polymer layer is impaired in carrying out evasive movements, even higher stresses of the same are to be expected.

According to the invention, it is now provided that a surface structure, onto which the coating is applied, is provided in the region of the respective curved section on the respective concave side, and/or that a layer made of a placeholder material is arranged between the coating and the respective curved section of the base body, so as to reduce crack formation of the coating.

The stent is preferably a biodegradable stent. This means that the implanted stent is designed to break down in the patient's body within an, in particular defined, period of time. The coating can include at least one substance that, when the stent is implanted, is released into the patient's body and constitutes a drug, for example to prevent restenosis (known as drug-eluting stent or DES). In this regard, primarily sirolimus and the various derivatives thereof, and paclitaxel, should be mentioned. The coating can further be designed to prolong the duration of the biodegradation process of the base body in a defined manner. In a preferred embodiment the biodegradable stent is a stent made of one piece.

According to one embodiment of the invention, it is provided that the stent can be transferred from an initial state into an expanded state by expanding the stent in the radial direction. The respective radial direction is perpendicular to the axial direction.

According to one embodiment of the invention, it is provided that the placeholder material is designed to be free-flowing in the implanted state.

According to one embodiment, it is further provided that the placeholder material is free-flowing at body temperature (i.e. 37° C.) and that the placeholder material is solid at room temperature (in particular 20° C.).

The surface structure of the respective concave side is used to increase the effective surface, whereby comparatively more coating material can be applied, or is applied, to the respective concave side along an extension direction of the respective curved section. During expansion or dilation, the coating may detach in this area, and instead of the coating being strained, which can result in crack formation, stretching occurs in the extension direction of the respective curved section by smoothing the additional or undulated coating material. As an alternative or in addition, the placeholder material that the casing formed by the coating is able to move, during expansion of the stent, in the radial direction transversely to the extension direction of the respective curved section, wherein the placeholder material flows around the respective curved section, so that excessively high strain of the coating along the extension direction is avoided.

Moreover, according to one embodiment of the invention it is provided that the respective surface structure is formed by a surface having alternating elevations and depressions.

The surface structure is preferably a regular or ordered surface structure, wherein, for example, a width of the respective depression and/or a width of the respective elevation (at half the height or depth) along said extension direction is in the range of 1% to 200%, preferably of 1% to 90% of the web width, and more preferably in the range of 15 to 65% of the web width. Furthermore, a height of the respective elevation (relative to a base point of the elevation) and/or a depth of the respective depression are in the range of 1% to 100%, and preferably of 1% to 35% of the web width.

According to one embodiment of the invention, it is provided that the respective surface is undulated or wave-like. Advantageously, the coating has an undulated or wave-like form as well which does not rip upon stretching and loosening from the surface of the scaffold comparable to way of working of a spring or coil.

According to one embodiment of the invention, it is further provided that the respective surface is formed by a surface of the concave side of the at least one curved section of the respective strut.

According to an alternative embodiment of the invention, it is further provided that the respective surface is formed by a surface of a separate element, wherein the respective separate element is fixed on the concave side of the at least one curved section of the respective strut.

According to one embodiment of the invention, it is further provided that, in the initial state of the stent, the respective layer made of the placeholder material is arranged predominantly or completely on the concave side of the at least one curved section of the respective strut.

As an alternative, it may be provided that, in the initial state of the stent, the respective layer of the placeholder material surrounds the curved section of the respective strut in the cross-section.

The layers of the placeholder material on the concave sides or around the respective curved section of the struts can also be formed so as to be cohesive, i.e. to be joined to one another.

According to one embodiment, the entire base body is surrounded by a cohesive layer made of the placeholder material. This means that the layers form a cohesive layer surrounding the base body. In particular, said coating is then applied onto this layer.

According to one embodiment of the invention, it is further provided that the respective layer of the placeholder material is designed, when the stent is implanted and when the stent is being transferred into the expanded state, to flow, or to be displaced, at least partially from the convex side onto the concave side of the at least one curved section of the respective strut. In this way, excessive stress/strain of the coating in the region of the curved section of the struts can be prevented, which significantly decreases the risk of crack formation.

According to one embodiment of the invention, it is further provided that the respective strut includes a plurality of mutually connected curved sections, so that the respective strut has a meander-like progression, wherein the respective curved section has a concave side and a convex side, and wherein the stent is designed so as to be expandable in the radial direction, whereby a curvature of the respective curved section is decreased when the stent is expanded in the radial direction, and wherein the stent can be transferred from an initial state into an expanded state, wherein a curvature of the respective curved section is decreased in the expanded state compared to the initial state, and wherein a surface structure, onto which the coating is applied, is provided in the region of the respective curved section on the respective concave side, and/or a layer made of a placeholder material is arranged between the coating and the respective curved section of the base body, so as to reduce crack formation of the coating.

The respective surface structure can, in turn, be designed according to one of the above-described embodiments. The same applies with respect to the respective layer of the placeholder material. This means that a placeholder material can be provided in the above-described manner on the respective concave side or on the respective curved section.

According to one embodiment of the present invention, it is further provided that the respective strut extends around the periphery in a circumferential direction of the base body.

According to one embodiment of the present invention, it may further be provided that the respective strut is connected to a strut adjoining in the axial direction by at least one web extending along the axial direction. Preferably, the respective circumferential and in particular meander-shaped strut is connected to an adjoining strut in each case by two webs.

According to one embodiment of the invention, it is further provided that the placeholder material is formed by one of the following substances or includes one of the following substances: a hydrogel, a thermoreversible hydrogel, a thermoreversible hydrogel that is liquid at body temperature and solid at room temperature. An example for a hydrogel is polyacrylamide. Further, a suitable placeholder material is a mixture of polyethylene glycol/poly-L-lactide.

According to one embodiment of the invention, it is further provided that the placeholder material is separated from the coating by a separating layer.

According to one embodiment of the invention, the separating layer can include one of the following substances or be formed of one of the following substances: magnesium stearate, zinc stearate, lithium stearate.

According to one embodiment of the invention, it is further provided that the coating is formed by one of the following substances or includes one of the following substances: a polymer such as polylactide, and in particular poly-L-lactide, polylactide-co-glycolide, polycaprolactone or combinations thereof (blends, copolymers). Hence, it is preferred that the coating is also biodegradable as is the scaffold.

According to one embodiment of the invention, it is further provided that the base body is formed of one of the following substances or includes one of the following substances: a magnesium alloy, e.g. alloyed with rare earths (e.g., WE43) or magnesium aluminum alloys.

In particular according to one embodiment, the magnesium alloy is selected in such a way that the base body is biodegradable or can be broken down in the body of the patient (in particular over a defined period of time).

A further aspect of the present invention relates to a method for producing a stent according to the invention, wherein the base body of the stent is provided, and wherein the respective curved section is provided with the respective surface structure on the concave side, and/or wherein a layer of a placeholder material is applied onto the respective curved section of the base body, and wherein subsequently the coating is applied.

The surface structures or said layers can be arranged or formed in the above-described manners in the method.

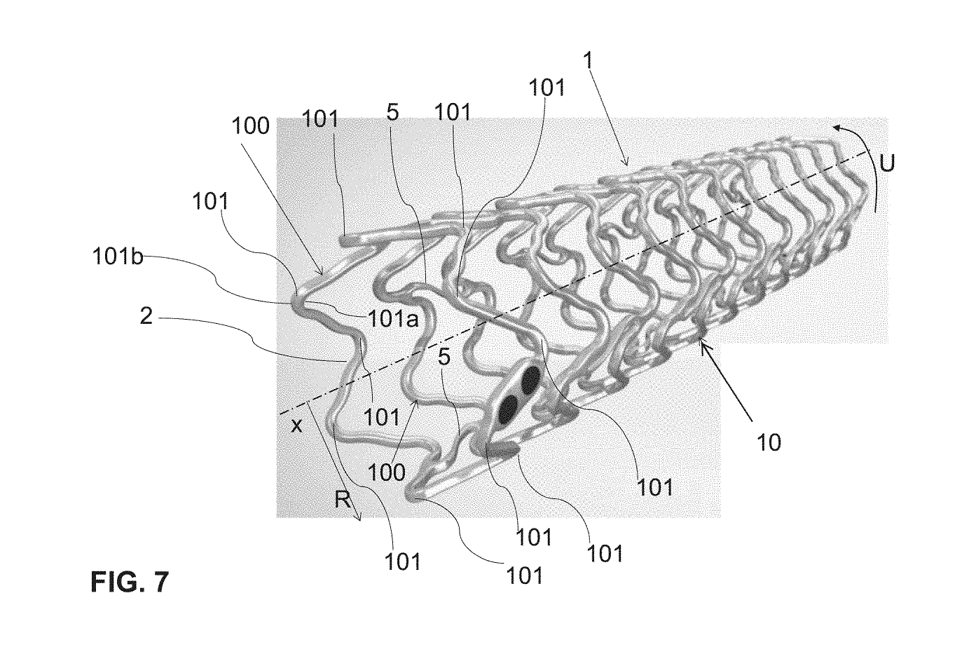

FIG. 7 shows an embodiment of a stent 1 according to the invention, wherein the stent 1 has a base body 10 that is coated with a coating 2 and extends in an axial direction x, wherein the base body 10 includes a plurality of mutually connected struts 100, wherein the respective strut 100 has at least one curved section 101, and preferably multiple such sections 101, wherein the respective curved section 101 has a concave side 101a and a convex side 101b, and wherein the stent 1 can be transferred from an initial state into an expanded state, wherein a curvature of the respective curved section 101 is reduced in the expanded state compared to the initial state. According to the invention, it is now provided that a surface structure 3, onto which the coating 2 is applied, is provided in the region of the respective curved section 101 on the respective concave side 101a, and/or that a layer 4 of a placeholder material is arranged between the coating 2 and the respective curved section 101 of the base body, 10 so as to reduce crack formation of the coating 2.

The respective strut 100 of the base body 10 can extend around the periphery in the circumferential direction U of the base body 10, wherein the curved sections 101 are designed so as to form, based on the axial direction x, alternatingly arranged minima and maxima, that is form a meander-shaped structure. Moreover, the respective strut 100 can be connected to a strut 100 adjoining in the axial direction x, for example by two webs 5.

The provision according to the invention of surface structures 3 and/or placeholder material layers 4 is carried out in particular due to the following problem.

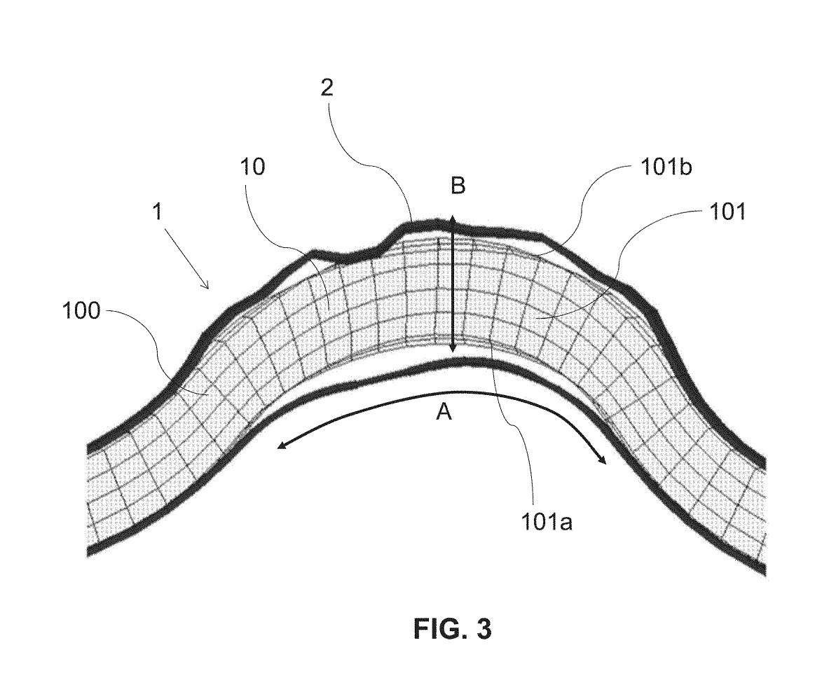

Bending open the curved sections 101, which are designed in particular as meander curves 101, during dilation or expansion of the stent 1 in the radial direction R creates two kinds of tensile stresses in the coating 2, which are shown in FIG. 3, namely on the one hand due to strain of the coating material 2 on the respective concave side or flank 101a of the curved section 101, i.e., on the pulling side of the bending structure in direction A, and on the other hand by the endeavor of the coating 2 to detach from the respective concave side 101a (B), which is impeded by the fact that the coating 2 surrounds the entire strut 100 or the base body 10 in each case. The two mechanisms are in particular coupled: if a greater extent of detachment from the respective concave side 101a is possible (without excessive tensile stress in the region B), the tensile stress along the direction A will be lower.

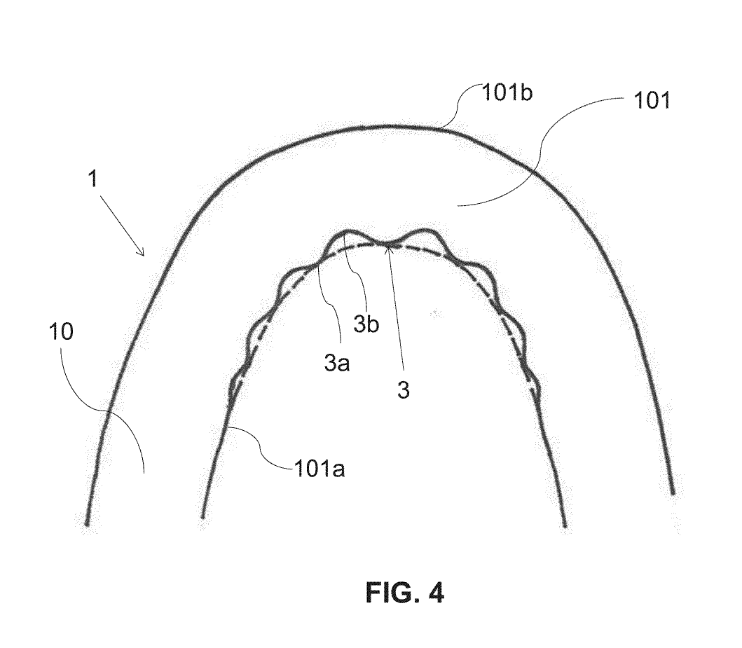

One option for ensuring a greater non-deformed material length of the coating 2 along the direction A is shown in FIG. 4. Here, the respective concave side 101a is provided with an undulated surface 3. During the application of the coating 2, the coating 2 on the respective concave side 101a becomes seated against the undulated contour or surface 3, which results in more material along the direction A (see FIG. 3) compared to the usual progression of the contour (FIG. 4: dotted). During the expansion in the radial direction R, the coating 2 detaches in particular in this region, and stretching in the direction A results from the smoothing of the undulations, instead of due to strain of the coating material 2. Instead of directly using an undulated geometry of the scaffold base body 10, the undulated contour or surface 3 can also be generated by using a separate element, for example a wax-like material.

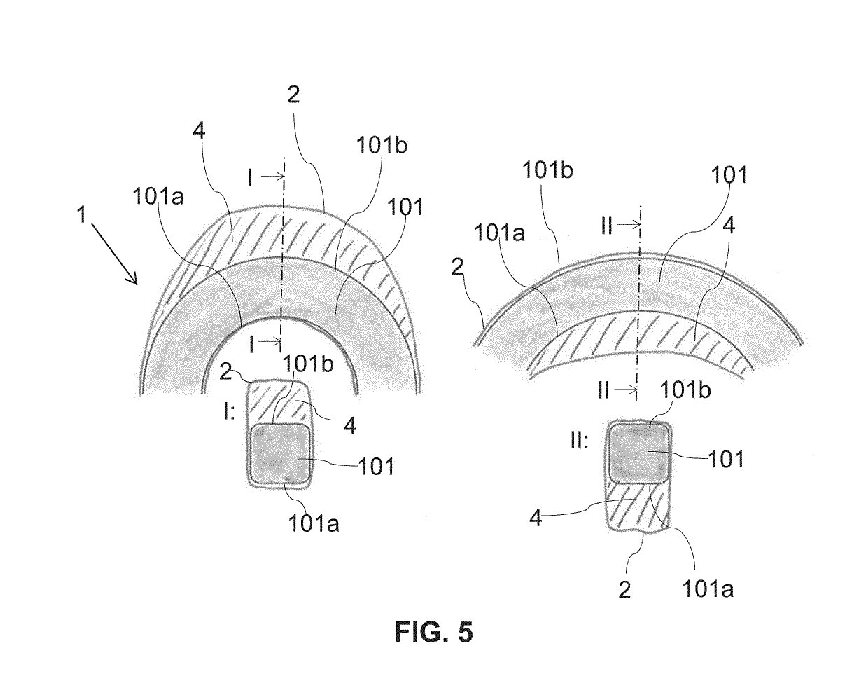

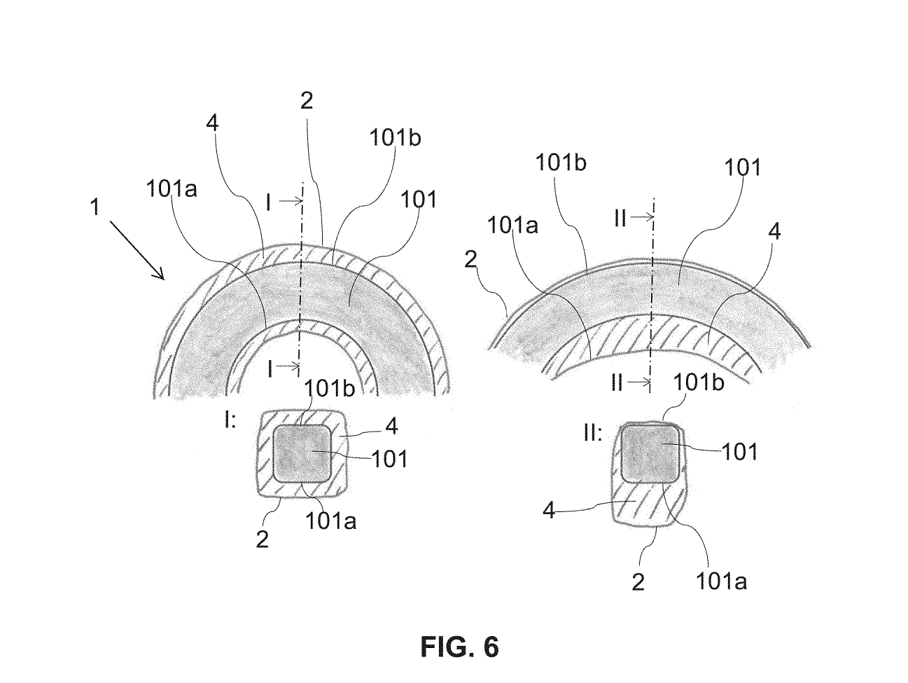

A further embodiment of the invention, which is shown in FIGS. 5 and 6, uses an approach in direction B (see FIG. 3). Prior to the coating 2, a placeholder material 4 is applied onto the base body 10, which ensures a distance between the base body material 10 and the coating 2 in the desired locations and which, in particular, has properties (for example, soft, and preferably viscous) that allow the placeholder material to flow between the coating 2 and the base body 10 around the respective curved section 101.

From a mechanical point of view, the embodiment shown in FIG. 5 is particularly advantageous. The placeholder material is deliberately applied only onto the convex sides 101a of the curved sections or meander curves 101 in the form of a respective layer 4 (see FIG. 5, left side). During the dilation, the placeholder material 4 flows around the respective curved section 101 of the particular strut 100 (see FIG. 5, right side), so that the entire coating 2 is able to move in direction B (see FIG. 3), without excessively high strain occurring in the coating in direction A (see FIG. 3).

FIG. 6 further shows an alternative embodiment which, compared to the embodiment shown in FIG. 5, has the advantage of a simplified production of the stent 1 since here a deliberate application of the placeholder material 4 onto the concave sides 101a is circumvented by providing the placeholder material, or a layer 4 made thereof, on all sides of the respective curved section 101 of the base body 10, and in particular on the concave and convex sides 101a, 101b, so that the respective section 101 or the respective strut 100, in the cross-section, is surrounded by the placeholder material or a corresponding layer 4 (see FIG. 6, left side). This layer 4 can be applied onto the base body 10, for example, by a dipping or spraying method. In this case as well, the placeholder material flowing around the respective strut 100 or the respective section 101 (FIG. 6, right side) ensures that the coating 2 is able to move in direction B (see FIG. 3) when the stent is being expanded in the radial direction, without excessively high strain occurring in the coating 2 in direction A (see FIG. 3).

Preferably, the placeholder material used is a thermoreversible hydrogel, for example polyacrylamide or polyethylene glycol/poly-L-lactide. Such hydrogels are characterized by gelling in certain temperature ranges, i.e. have a certain strength, while becoming liquid again in other temperature ranges. Preferably, the placeholder material or hydrogel according to the invention has a composition so as to be solid at room temperature and liquid at body temperature. An adaptation of the corresponding transition temperatures may also take place, for example, by selecting one or more hydrogels from the plurality of possible gels and by adjusting the mixing ratio of the gels.

A placeholder material liquefying at body temperature also serves as a separating agent between the coating 2 and the base body 10, counteracting undesirable local adhesions. For example, magnesium stearate can be used as a separating layer between the placeholder material and the coating 2 (in particular polymer), for example to avoid mixing.

In particular by reducing or avoiding the crack formation in the coating 2 of the base body 10 of the stent 1, which is made in particular of a magnesium alloy, the present invention allows the scaffolding time of the stent 1 to be prolonged. Using the solution according to the invention, this is possible, in particular, using the known polymer materials that are not critical from a regulatory point of view for the coating 2.

It will be apparent to those skilled in the art that numerous modifications and variations of the described examples and embodiments are possible in light of the above teaching. The disclosed examples and embodiments may include some or all of the features disclosed herein. Therefore, it is the intent to cover all such modifications and alternate embodiments as may come within the true scope of this invention.

What is claimed is:

1. A stent comprising a base body coated with a coating and extending in an axial direction, the base body comprising a plurality of mutually connected struts, each strut comprising at least one curved section which has a concave side and a convex side, the stent being expandable from an initial state into an expanded state, a curvature of the curved section being reduced in the expanded state compared to the initial state, and a surface structure on the concave side and/or a layer of a placeholder material between the coating and the curved section.

2. The stent according to claim 1, wherein the surface comprises alternating elevations and depressions.

3. The stent according to claim 1, wherein the respective surface is an undulating surface.

4. The stent according to claim 1, comprising a surface structure on the concave side of the at least one curved section.

5. The stent according to claim 1, wherein the respective surface structure is formed from additional material on the base body.

6. The stent according to claim 1, wherein in the initial state of the stent, the placeholder material is arranged predominantly or completely on the convex side of the at least one curved section.

7. The stent according to claim 1, wherein in the initial state of the stent, the placeholder material surrounds the at least one curved section.

8. The stent according to claim 1, wherein the placeholder material is a material structured to flow at least partially from the convex side onto the concave side of the at least one curved section during expansion.

9. The stent according to claim 1, wherein the struts comprise a plurality of mutually connected curved sections, so that the respective strut has a meander-like progression, each respective curved section having a concave side and a convex side, a curvature of each respective curved section being reduced in the expanded state compared to the initial state, and the surface structure, onto which the coating is applied, being provided on the respective concave side of each respective curved section, and/or a of the placeholder material being arranged between the coating and each respective curved section.

10. The stent according claim 1, wherein the struts extend around the periphery in a circumferential direction of the base body.

11. The stent according to claim 1, wherein each strut is connected to an adjoining strut by at least one web extending along the axial direction.

12. The stent according to claim 1, wherein the placeholder material is formed by one of the following substances or comprises one of the following substances: a hydrogel, a thermoreversible hydrogel, a thermoreversible hydrogel that is liquid at body temperature and solid at room temperature

13. The stent according to claim 12, wherein the thermoreversible hydrogel is polyacrylamide or polyethylene glycol/poly-L-lactide.

14. The stent according to claim 1, wherein the placeholder material is separated from the coating by a separating layer.

15. The stent according to claim 1, wherein the coating is formed by or comprises a polymer.

16. The stent according to claim 15, wherein the polymer is polylactide.

17. The stent according to claim 15, wherein the polymer is poly-L-lactide, polylactide-co-glycolide, polycaprolactone or combinations thereof.

18. The stent according to claim 1, wherein the base body is formed of one of the following substances or comprises one of the following substances: magnesium or a magnesium alloy.
